(19) United States
(12) Patent Application Publication
(10) Pub. No.: US 2002/0138860 A1
(43) Pub. Date: Sep. 26, 2002

(54) Novel uses of mammalian CCR6 receptors and related reagents

(76) Inventors: Donald Cook, Cranford, NJ (US); Sergio A. Lira, Chatham, NJ (US); Satwant K. Narula, West Caldwell, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 10/102,468

(22) Filed: Mar. 20, 2002

Related U.S. Application Data
(62) Division of application No. 09/656,927, filed on Sep. 7, 2000.
(60) Provisional application No. 60/152,866, filed on Sep. 8, 1999.

(51) Int. Cl. A01K 67/00; A61K 39/395; A61K 31/00

(52) U.S. Cl. 800/8; 424/144.1; 514/1

(57) ABSTRACT

Compositions and methods for using mammalian CCR6 and MIP-3α proteins to treat an abnormal physiological condition in an individual. The methods comprise administering a therapeutically effective amount of CCR6 or MIP-3α alone, or in combination with other therapeutic reagents; or a CCR6 or MIP-3α antagonist. Genetically-engineered animals and their use as models of molecular mechanism are also provided.

### FIELD OF THE INVENTION

This application claims the benefit of U.S. Provisional Application No. 60/152,866 filed Sep. 8, 1999. The present invention relates to methods of using proteins which function in controlling development, differentiation, trafficking, and physiology of mammalian cells, e.g., cells of a mammalian immune system. In particular, it provides methods of using proteins and mimetics which regulate cellular mucosal immunity.

The present invention also relates to genetically engineered non-human animals and their use as molecular models in the study of the CCR6 chemokine receptor and molecules affected by CCR6's action.

### BACKGROUND OF THE INVENTION

The immune system of vertebrates consists of a number of organs and several different cell types. See, e.g., Paul (ed. 1997) Fundamental Immunology (4th ed.) Raven Press, New York. The migration and activation of lymphocytes in homeostasis and during inflammatory responses is mediated largely by chemokines, a large family of chemotactic proteins. See, e.g., Schall et al., 1994, Current Opinion in Immunology 6:865-873; Bacon et al., 1996, Int. Arch. Allergy and Immunol. 109:97-109; Springer et al., 1994, Cell 76:

301-314; Rollins et al., 1997, Blood 90:909-928. Chemokines are secreted by activated leukocytes themselves, and by stromal cells including endothelial cells and epithelial cells upon inflammatory stimuli. See Oppenheim, 1993, Adv. Exp. Med. Biol. 351:183-186; Schall, et al., 1994, Curr. Opin. Immunol. 6:865-873; Rollins, 1997, Blood 90:909-928; Baggiolini, et al., 1994, Adv. Immunol. 55:97-179. Responses to chemokines are mediated by seven transmembrane spanning G-protein-coupled receptors (Rollins, 1997, Blood 90:909-928; Premack, et al., 1996, Nat. Med. 2:1174-1178; Murphy, P. M. 1994, Ann. Rev. Immunol. 12:593-633).

The roles and mechanisms of action of chemokines and other signaling molecules which induce, sustain, or modulate the various physiological, developmental, or proliferative states of the cells of the immune system are poorly understood. Clearly, the immune system and its response to various stresses has relevance to medicine, e.g., clearance of cellular or other materials after injury, infectious diseases, cancer related responses and treatment, and allergic and transplantation rejection responses. See, e.g., Thorn, et al. Harrison's Principles of Internal Medicine McGraw/Hill, New York; Ziegler, et al. (ed. 1997) Growth Factors and Wound Healing: Basic Science and Potential Clinical Applications Springer Verlag; Clark (ed. 1996) The Molecular and Cellular Biology of Wound Repair Plenum; and Peacock (1984) Wound Repair Saunders.

Medical science relies, in large degree, to appropriate recruitment or suppression of the immune system in effecting cures for insufficient or improper physiological responses to environmental factors. However, the lack of understanding of how the immune system is regulated or differentiates has blocked the ability to advantageously modulate the immunological mechanisms to biological challenges, i.e., response to biological injury. Medical conditions characterized by abnormal or inappropriate regulation of the development or physiology of relevant cells thus remain unmanageable. The discovery and characterization of specific regulatory pathways and their physiological effects will contribute to the development of therapies for a broad range of degenerative or other conditions which affect the biological system, immune cells, as well as other cell types. Understanding the role of immune cells and their overall function in the development of various inflammatory conditions has been hampered by the lack of in vivo models. The present invention provides solutions to some of these and many other problems.

### SUMMARY OF THE INVENTION

The present invention is based, in part, upon the discovery of the physiological role of the chemokine receptor CCR6 and its ligand MIP-3α in various models of immune response. In particular, the role of CCR6 has been elucidated in pathways involved in inflammation in the gut, and in allergic or other respiratory diseases involving pulmonary inflammation. The invention also relates to the identification of a model system to study the role and function of CCR6 receptors through the use of genetically engineered animals which lack a functional CCR6 gene.

The present invention provides methods of modulating the trafficking or activation of a leukocyte in an animal, the methods comprising contacting leukocytes in the animal with a therapeutic amount of an agonist of a mammalian CCR6 or MIP-3α protein; or an antagonist of a mammalian CCR6 or MIP-3α protein. Preferred embodiments include where: the mammalian CCR6 or MIP-3α protein is a primate protein. Further embodiments include where the antagonist is an antibody which binds to the mammalian CCR6 or MIP-3α or where the antagonist is a small molecule inhibitor. Certain embodiments include where the leukocytes include a B cell or a T cell or a dendritic cell, or where the animal exhibits signs or symptoms of an inflammatory or leukoproliferative condition. Preferred embodiments include where the sign or symptom is in mucosal tissue, e.g. in pulmonary or skin tissue; neural tissue; lymphoid tissue; myeloid tissue; pancreas; gastrointestinal tissue; thyroid tissue; muscle tissue; or collagenous tissue.

The methods of the invention include where the modulating is inhibiting function of the CCR6 receptor; and/or where the administering is the antagonist. Preferably, the antagonist is: an antibody which binds to the mammalian CCR6 or MIP-3α; a small molecule inhibitor that blocks the function of CCR6 or MIP-3α; or a mutein of the mammalian CCR6 or MIP-3α which competes with the mammalian MIP-3α in binding to a CCR6 receptor, but does not substantially signal. Certain embodiments include where the animal is experiencing signs or symptoms of an inflammatory condition or autoimmunity; asthma; tissue specific autoimmunity; degenerative autoimmunity; rheumatoid arthritis; atherosclerosis; multiple sclerosis; delayed hypersensitivities; skin grafting; psoriasis; a transplant; spinal injury; stroke; neurodegeneration; or ischemia. The administering may be in combination with: an anti-inflammatory agent including an anti-inflammatory cytokine such as IL-10; a cytokine agonist or antagonist; an analgesic; an anti-diarrheal agent; or a steroid.

Various other methods are provided where the modulating is enhancing function of the MIP-3α-CCR6 reaction, and/or the administering is the agonist. In a particular embodiment, the method is applied where the animal experiences signs or symptoms of an inflammatory disease such as ciliac disease (sprue). The administering will often be in combination with an anti-inflammatory agent including anti-inflammatory cytokines such as IL-10; a cytokine agonist or antagonist; an analgesic; or a steroid.

The invention also provides a genetically engineered non-human animal whose genome lacks a functional CCR6 gene, and methods for its use as a model for molecular mechanism.

### BRIEF DESCRIPTION OF THE DRAWING FIGURE

FIG. 1 illustrates CCR6 gene targeting. FIG. 1A depicts the CCR6 locus. Restriction sites are Hind III (H), Bam HI (B), and Not I (N). Sizes are in kilobases. The single CCR6 exon is shown by a black rectangle and the direction of transcription by an arrow. FIG. 1B shows the targeting construct. The neomycin resistance gene (neo) is flanked by 5.5-kb and a 1.1-kb DNA fragments and the herpes simplex thymidine kinase gene (HSV TK). The Not I-linearized plasmid was electroporated into AV3 ES cells. FIG. 1C provides the structure of the targeted locus. The sizes of the expected fragments are shown in kilobases. Arrowheads represent the primers used in a polymerase chain reaction to identify targeted ES cell clones. The probe used for Southern blot confirmation of targeted clones is shown.

### DETAILED DESCRIPTION OF THE PREFERRED EMBODIMENTS

I. General

CCR6 is a chemokine receptor that is expressed on human dendritic cells, memory T cells and on B cells [Zaballos et al., 1996, Biochemical and Biophysical Research Communications 227:846-853; Greaves et al., 1997, Journal of Experimental Medicine 186:837-844; Power et al., 1997, Journal of Experimental Medicine 186:825-835; Liao et al., 1999, Journal of Immunology 162:186-94]. The only known ligand for CCR6 is the chemokine MIP-3α, also known as larc or exodus. [Rossi et al., 1997, J. Immunology 158:1033-1036].

The CCR6 receptor was first cloned from human genomic DNA as an orphan receptor [Zaballos et al., 1996, Biochemical and Biophysical Research Communications 227:846-853]. Northern blot analysis has revealed that CCR6 is expressed mainly in spleen, lymph nodes, appendix, and fetal liver among various human tissues. [Baba et al., 1997, J. Biol. Chem. 272:14893-14898]. Among various leukocyte subsets, CCR6 mRNA has been detected in lymphocytes (CD4+ and CD8+ T cells and B cells) but not in natural killer cells, monocytes, or granulocytes. [Baba et al., 1997, J. Biol. Chem. 272:14893-14898].

To study the biological role of CCR6 in vivo, a mouse CCR6 cDNA was cloned from the mouse spleen. This was used to determine the expression of CCR6 in 19 different mouse tissues. Northern blot analysis of CCR6 RNA was conducted [Molecular Cloning, a Laboratory Manual, second edition, 1989, Sambrook, Fritch, Maniatis, Cold Spring Harbor Press, 10 Skyline Drive, Plainview, N.Y. 11803-2500]. The analysis revealed high levels of CCR6 mRNA in Peyer's patches, the spleen and lymph nodes, with lower levels in the thymus, testes and spinal cord. This expression profile is generally similar to that of human CCR6, which is expressed in the spleen, thymus, small intestine and peripheral blood leukocytes (PBLs) [Baba et al., 1997, J. Biol. Chem. 272:14893-14898]. To determine the expression of MIP-3α, mouse MIP-3α was cloned. See Rossi et al., 1997, J. Immunology 158:1033-36; Hieshima et al., 1997, J. Biol. Chem 272: 5846-53. Analysis of MIP-3α mRNA revealed that it too was highly expressed in Peyer's patches, with lower levels in the large intestine and spinal cord.

Peyer's patches are a major site of uptake and presentation of antigens from the small intestine. Both the small intestine and colon must maintain a balance between an immune response against microbial pathogens, and a tolerance to food antigens and symbiotic bacteria. The response to the many antigens present in the gut is mediated by lymphocytes situated in Peyer's patches, the intestinal epithelium, and the lamina propria, an area of loose connective tissue underlying the epithelium. Memory T cells from each of these sites have a propensity to return from the blood back to the gut, whereas naive T cells distribute to tissues randomly. See Mackay et al., 1992, European Journal of Immunology 22:887-895; Hamann et al., 1994, Journal of Immunology 152:3282-93. This mucosal-specific homing of lymphocytes is mediated in part by cell surface α4β7 integrins which interact with the mucosal addressin cell adhesion molecules (MadCAM) presented on the high endothelial venules (HEV) of Peyer's patches and on the post-capillary venules in the lamina propria. Chemokines and their receptors may also have a role in lymphocyte homing [Springer, T., 1994, Cell 76:301-314], and thereby contribute to homeostasis of the number and types of lymphocytes situated in the gut mucosa.

To study in detail the expression of CCR6 and MIP-3α in Peyer's patches, in situ hybridization studies were performed. At low magnification, MIP-3α RNA was evident near the boundary of the lumen of the small intestine and the Peyer's patch; very little MIP-3α message was seen within the interior of the Peyer's patch. Higher magnification revealed that MIP-3α expression was restricted almost exclusively to the epithelial cells lining the Peyer's patch. Very little expression of this chemokine was detected within the underlying epithelial dome of the Peyer's patch, or in the epithelium covering the villi of the small intestine.

Like MIP-3α, CCR6 message was also seen near the junction of the intestinal lumen and the Peyer's patch, but was more diffuse than that of MIP-3α. High magnification revealed that most of the CCR6 message was not within epithelial cells, but rather in the adjacent cells of the subepithelial dome. Thus, MIP-3α and CCR6 are expressed in separate but adjacent cell populations within the Peyer's patch.

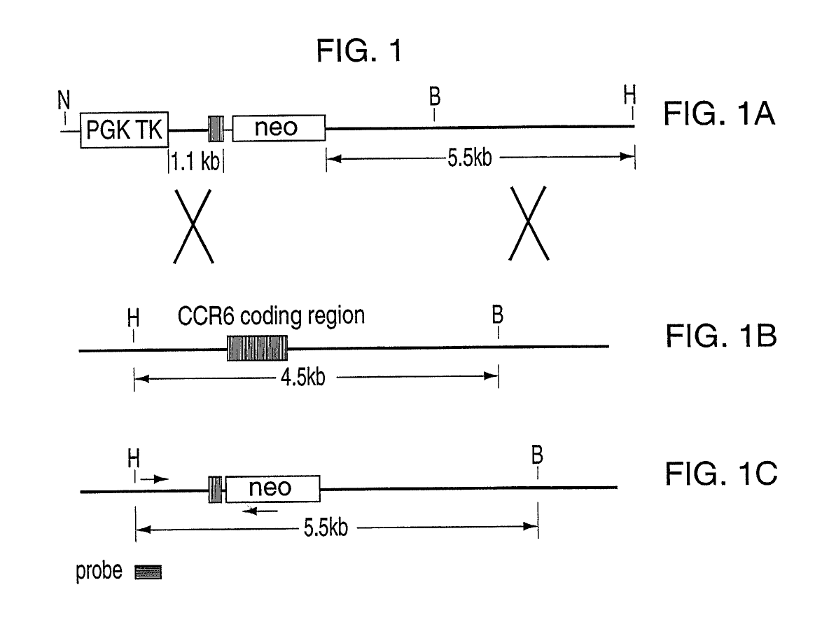

To directly investigate the biological role of CCR6 in the mouse, and in particular whether CCR6-MIP-3α interactions are involved in regulation of lymphocyte trafficking, a CCR6 targeting vector was generated. Gene targeting was used in embryonic stem (ES) cells derived from 129 Sv embryos [Teratocarcinomas and Embryonic Stem Cells: A practical Approach. ILR Press, Oxford, UK, 1987]. A targeting vector was constructed in which DNA from two regions of the CCR6 gene flanked a neomycin resistance gene (FIG. 1A). This DNA was transfected by electroporation into the ES cells, resulting in a deletion of the CCR6 gene in a manner similar to that described in Ramirez-Solis et al., 1993, Methods in Enzymology 225: 855.

Matings between CCR6 heterozygotes (+/−) obtained from ES cell-derived chimeras yielded offspring having a Mendelian proportion of +/+, +/− and −/− CCR6 genotypes. The −/− mice appeared healthy and bred well. To examine CCR6 mRNA expression in the −/− mice, RNA was prepared from the spleen of both +/+ and −/− mice. Northern blot analysis of this RNA with a probe corresponding to the coding region of CCR6 revealed that CCR6 mRNA was absent in the spleens of −/− mice (FIG. 1B). To confirm that the −/− mice were functionally null for CCR6, a binding assay using radiolabelled MIP-3α was performed. MIP-3α bound to membranes prepared from +/+ mice, but not to membranes from −/− mice, demonstrating that the mice were functionally null, and that no other receptor in the murine spleen binds MIP-3α with high affinity.

No abnormalities were apparent in the development of any major organs of the −/− mice, and all tissues appeared normal by light microscopy. Flow cytometric analysis of the leukocytes in spleen, blood, lymph nodes, thymus and bone marrow did not reveal differences between −/− and +/+ mice. In Peyer's patches, a very slight increase was seen in the number CD8⁺ T cells. In addition, a slight decrease was seen in the number of B cells. These differences in numbers of B and T cells in Peyers patches of wild type and CCR6 were very small and probably not significant. However, importantly, recent experiments have revealed that dendritic cells expressing the surface markers CD11c and CD11b are absent in the subepithelial dome (SED) of CCR6 −/− mice. Thus, CCR6 is required for the normal positioning of these cells in the SED. This finding suggests that the character of the immune response in the gut, and perhaps mucosal tissue in general, is affected by CCR6-mediated positioning of dendritic cells in Peyers patches.

To determine whether CCR6 deficiency results in a dysregulation of lymphocyte homeostasis in the intestine, flow cytometric analysis of the leukocytes in the intestinal epithelium and lamina propria of the colon and small intestine was performed [Rogler et al., 1998, Clinical and Experimental Immunology 112:205-215]. No differences between +/+ and −/− mice were seen in lymphocytes prepared from the colon. However, in the −/− mice, a larger number of intraepithelial lymphocytes (IEL) and lamina propria lymphocytes (LPL) was seen in the small intestine. This increased number of T cells was due to a cells staining for the T cell markers CD3, CD8α and the activation marker CD69⁺. The percentage of B cells in the gut is altered in −/− mice, although absolute numbers of these cells are unchanged.

There are two major lineages of T lymphocytes: one expresses the αβ T cell receptor (TCR αβ), and the other expresses TCRγδ. TCRγδ cells represent a minor fraction of the lymphocytes present in most peripheral lymphoid tissues, but comprise about half of the lymphocytes in the intestinal mucosa of mice. Goodman et al., 1988, Nature 333:855-858. To determine to whether the cells that were increased in the gut of the −/− mice expressed TCR α/βor TCR γδ, flow cytometric analysis of IELs and LPLs was performed. When total viable cells were gated, a significant increase in TCRαβ cells were seen in the −/− mice, but TCRγδ cells were unchanged. When cells having the forward and side scatter characteristics of lymphocytes were gated, most cells in the +/+ intestinal epithelium and lamina propria expressed TCR γδ. However, in the −/− mice, most cells expressed the TCR αβ. Thus, the increase in IELs and LPLs in the −/− mice is due to TCR αβ T cells, and not the mucosal tissue-restricted TCR γδ cells.

No foci of inflammatory cells were seen by light microscopic analysis of hematoxylin- and eosin-stained sections of the small intestine, and the structure of the villi appeared normal. To determine the pattern of distribution of the increased IELs and LPLs in the −/− mice, immunohistochemical staining of frozen sections was performed. For immunostaining, fresh frozen sections at 8-10 m were first fixed with ice-cold acetone for 10 minutes and air-dried. The sections were then blocked with sequential treatment of biotin-avidin blocking kit [Vector, Burlingame, Calif.], 3% H₂O₂, and 10% normal mouse serum (Vector) for 15 minutes each. Anti-CD8b antibody at 0.5 (g/ml was incubated for 1 hr at r.t. followed by incubation with a biotinylated mouse anti-rat IgG1/IgG2 (Pharmigen) for 30 minutes. A Vectastain Elite ABC kit (Vector) was then used following the instruction from a manufacturer. The tissue sections were finally stained with diaminobenzidine (Vector) and counterstained with Hematoxylin. All dilution was made with a buffer containing 1XPBS and 0.1% SDS.

These experiments confirmed the increased number of CD8⁺ T cells, and also revealed that these cells were not clustered in discrete foci. Rather, they were distributed throughout the small intestine in a diffuse pattern. This low-grade pathology in the small intestine is similar to that proposed for humans that suffer from Celiac disease (sprue). These individuals are thought to have normally structured villi in the small intestine, but have a high IEL count that results from sensitization by the wheat-derived protein gluten. See Ferguson et al., 1996, Ann. N. Y Acad. Sci. 778:202-16. In a proportion of these individuals, the combination of sensitizing antigen and activated mucosal T cells results in severe eneropathy and malabsorption that may persist for several weeks after gluten has been withdrawn from the diet. It is unclear whether the increase in activated IELs seen in the −/− mice is associated with the presence of a sensitizing antigen in their diet.

The finding of dysregulated homeostasis in the intestinal mucosa prompted investigation of the immune response in the mucosa of the lung. Mice that are sensitized to ovalbumin undergo an allergic inflammatory response in the lung upon challenge with aerosolized protein. See Kung et al., 1994, International Archives of Allergy and Immunology 105:83-90. The cellular infiltrate in the bronchioalveolar lavage (BAL) of these mice consists largely of eosinophils, and this response is dependent on both functional T lymphocytes and dendritic cells. Garlisi et al., 1995, Clinical Immunology 75:75-83. Both +/+ and −/− mice were challenged in this type of experiment. Compared to unsensitized +/+ mice, the sensitized +/+ mice had a large increase in the total numbers of cells, and total eosinophils in the BAL 48 h following the aerosol challenge. However, a marked reduction was seen in total cells and total eosinphils of the challenged −/− mice compared to the +/+ control mice (p<0.05). This decreased pulmonary infiltrate in the −/− mice was not due to an inability to mount an immune response because the ovalbumin-specific levels of circulating antibodies in +/+ and −/− mice were indistinguishable. Thus, the absence of CCR6 impacts on the inflammatory-specific component of this allergic response.

If this hypothesized role of the CCR6 interaction holds true, manipulation of this interaction can have important clinical implications. In settings where enhanced CCR6-MIP-3α interaction is desired, agonists of CCR6 or MIP-3α would be beneficial. Conversely, in situations where the CCR6-MIP-3α interaction should be suppressed, a CCR6 or MIP-3α agonist could be useful.

The CCR6 KO mice of the present invention can now be used in studies of allergy, general immunity, viral immunity, and autoimmune diseases. Examples of models which can be used include those described in Swanson et al., 1985, J. Allergy & Clin. Immunology 76(5):724-29; Stevens et al., 1999, J. Immunol. 162(12):7501-9; Kung et al., 1994, International Archives of Allergy & Immunol. 105:83-90; Campbell et al., 1998, J. Immunol. 161(12):7047-53.

The descriptions below are directed, for exemplary purposes, to primate, e.g., a human, or rodent, e.g., mouse or rat CCR6 and MIP-3α, but are likewise applicable to related embodiments from other species. Thus, conditions known to be mediated by or related to lymphocyte trafficking may be regulatable using these reagents.

II. Nucleic Acids

General description of nucleic acids, their manipulation, and their uses (including, e.g., complementary and antisense nucleic acids) are provided in the following references:

GenBank Accession No. U45984, U60000 (CCR6) GenBank Accession No. U90447 (MIP-3α); International Patent Appln. No. WO 98/01557; Zaballos et al., 1996, Biochem Biophys. Res. Comm. 227:846-853 (CCR6); Hieshima et al., 1997, J. Biol. Chem. 272(9):5846-5853 (MIP-3α); Rossi et al., 1997, J. Immunology 158:1033-1036 (MIP-3α) McCaughein et al., “Transgenic Animals” in Roitt (ed.) Encyclopedia of Immunology Academic Press, San Diego, pp. 1502-1504; Travis (1992) Science 256:1392-1394; Kuhn, et al. (1991) Science 254:707-710; Capeechi (1989) Science 244:1288; Robertson (ed. 1987) Teratocarcinomas and Embryonic Stem Cells: A Practical Approach IRL Press, Oxford; Rosenberg (1992) J. Clinical Oncology 10:180-199; Cournoyer and Caskey (1993) Ann. Rev. Immunol. 11:297-329; Wetmur and Davidson (1968) J. Mol. Biol. 31:349-370; Weintraub (1990) Scientific American 262:40-46; Jaroszewski and Cohen (1991) Advanced Drug Delivery Reviews 6:235-250; Akhtar, et al. (1992) pages 133-145 in Erickson and Izant (eds.) Gene Regulation: Biology of Antisense RNA and DNA Raven Press, New York; Zhao, et al. (1994) Blood 84:3660-3666; Misquitta, et al. (1999) Proc. Nat'l Acad. Sci. USA 96:1451-1456; and Treco WO96/29411, each of which is incorporated by reference. Additional aspects will be apparent to a person having ordinary skill in the art in light of the teachings provided herein.

III. Purified CCR6 and MIP-3α protein

General descriptions of proteins and polypeptides in pharmaceutical or biochemical contexts can be found, e.g., in: Goodman, et al. (eds. 1990) Goodman & Gilman's: The Pharmacological Bases of Therapeutics (8th ed.) Pergamon Press; Avis, et al. (eds. 1993) Pharmaceutical Dosage Forms: Parenteral Medications Dekker, New York; Lieberman, et al. (eds. 1990) Pharmaceutical Dosage Forms: Tablets Dekker, New York; Lieberman, et al. (eds. 1990) Pharmaceutical Dosage Forms: Disperse Systems Dekker, New York; Freifelder (1982) Physical Biochemistry (2d ed.) W. H. Freeman; Cantor and Schimmel (1980) Biophysical Chemistry, parts 1-3, W. H. Freeman & Co., San Francisco. Specific descriptions of CCR6 are found, e.g., in WO98/01557 (human). Specific descriptions of MIP-3α are found, e.g., in WO98/01557 (mouse). Recombinant methods for making the proteins are well known.

Preparation of fragments by synthetic methods, or by biochemical cleavage of natural or recombinant forms, are available.

IV. Making CCR6 and MIP-3α proteins; Mimetics

DNA which encodes the CCR6 or MIP-3α proteins or fragments thereof can be obtained by chemical synthesis, screening cDNA libraries, or by screening genomic libraries prepared from a wide variety of cell lines or tissue samples.

This DNA can be expressed in a wide variety of expression systems as described in, e.g., U.S. Ser. No. 08/250,846; U.S. Ser. No. 08/177,747; U.S. Ser. No. 08/077,203; PCT/US95/00001; Kaufman, et al. (1985) Molec. and Cell. Biol. 5:1750-1759; Pouwels, et al. (1985 and Supplements) Cloning Vectors: A Laboratory Manual, Elsevier, N.Y., Rodriguez, et al. (eds. 1988) Vectors: A Survey of Molecular Cloning Vectors and Their Uses, Buttersworth, Boston, Mass.; Rodriguez and Denhardt (eds.) Vectors: A Survey of Molecular Cloning Vectors and Their Uses Buttersworth, Boston, Chapter 10, pp. 205-236; Okayama, et al. (1985) Mol. Cell Biol. 5:1136-1142; pMC1neo Poly-A, see Thomas, et al. (1987) Cell 51:503-512; O'Reilly, et al. (1992) Baculovirus Expression Vectors: A Laboratory Manual Freeman and Co., CRC Press, Boca Raton, Fla.; Low (1989) Biochem. Biophys. Acta 988:427-454; Tse, et al. (1985) Science 230:1003-1008; and Brunner, et al. (1991) J. Cell Biol. 114:1275-1283; each of which is incorporated herein by reference.

Now that the various CCR6 and MIP-3α proteins have been characterized, fusion polypeptides, fragments, or derivatives thereof can be prepared by conventional processes for synthesizing peptides. These include processes such as are described in Stewart and Young (1984) Solid Phase Peptide Synthesis Pierce Chemical Co., Rockford, Ill.; Bodanszky and Bodanszky (1984) The Practice of Peptide Synthesis Springer-Verlag, New York; Bodanszky (1984) The Principles of Peptide Synthesis Springer-Verlag, New York; and Merrifield, et al. (1963) in J. Am. Chem. Soc. 85:2149-2156; each of which is incorporated herein by reference. Additional aspects will be apparent to a person having ordinary skill in the art in light of the teachings provided herein.

V. Physical Variants

Proteins or peptides having substantial amino acid sequence homology with the amino acid sequence of the CCR6 or MIP-3α proteins are also contemplated. The variants include species or allelic variants. Homology, or sequence identity, is defined in, e.g., U.S. Ser. No. 08/250,846; U.S. Ser. No. 08/177,747; U.S. Ser. No. 08/077,203; PCT/US95/00001; Needleham, et al. (1970) J. Mol. Biol. 48:443-453; Sankoff, et al. (1983) Chapter One in Time Warps, String Edits, and Macromolecules: The Theory and Practice of Sequence Comparison Addison-Wesley, Reading, Mass.; software packages from NCBI, NIH; and the University of Wisconsin Genetics Computer Group, Madison, Wis.

The isolated DNA encoding a CCR6 or MIP-3α protein can be readily modified as described in, e.g., Sambrook, et al. (1989); Ausubel, et al. (1987 and Supplements); Cunningham, et al. (1989) Science 243:1330-1336; O'Dowd, et al. (1988) J. Biol. Chem. 263:15985-15992; and Carruthers (1981) Tetra. Letts. 22:1859-1862; each of which is incorporated herein by reference. Additional methods will be apparent to a person having ordinary skill in the art in light of the teachings provided herein.

VI. Functional Variants

The blocking of the physiological interaction between CCR6 and its ligand, MIP-3α, may result from the inhibition of binding of the ligand to the receptor by a variant of natural MIP-3α or antibody to MIP-3α, or by a variant of natural CCR6 or antibody to CCR6.

Methods for making such a variant are described in, e.g., Godowski, et al. (1988) Science 241:812-816; Beaucage and Carruthers (1981) Tetra. Letts. 22:1859-1862; Sambrook, et al. (1989) Molecular Cloning: A Laboratory Manual (2d ed.) Vols. 1-3, Cold Spring Harbor Laboratory; Merrifield (1963) J. Amer. Chem. Soc. 85:2149-2156; Merrifield (1986) Science 232: 341-347; Atherton, et al. (1989) Solid Phase Peptide Synthesis: A Practical Approach, IRL Press, Oxford; Cunningham, et al. (1989) Science 243:1339-1336; O'Dowd, et al. (1988) J. Biol. Chem. 263:15985-15992; and Lechleiter, et al. (1990) EMBO J. 9:4381-4390; each of which is incorporated herein by reference. Additional methods will be apparent to a person having ordinary skill in the art in light of the teachings provided herein.

VII. Specific Binding Compositions

A. Antibodies

The present invention provides for the use of an antibody or binding composition which specifically binds to a CCR6, preferably mammalian, e.g., primate, human, cat, dog, rat, or mouse. Antibodies can be raised to various CCR6 proteins, including individual, polymorphic, allelic, strain, or species variants, and fragments thereof, both in their naturally occurring (full-length) forms or in their recombinant forms. Additionally, antibodies can be raised to these proteins in both their native (or active) forms or in their inactive, e.g., denatured, forms. Anti-idiotypic antibodies may also be used.

A number of immunogens may be selected to produce antibodies specifically reactive, or selective for binding, with CCR6 proteins. Recombinant protein is a preferred immunogen for the production of monoclonal or polyclonal antibodies. Naturally occurring protein, from appropriate sources, e.g., primate, rodent, etc., may also be used either in pure or impure form.

Synthetic peptides, made using the protein sequences described herein, may also be used as an immunogen for the production of antibodies to the proteins. Recombinant protein can be expressed and purified in eukaryotic or prokaryotic cells as described, e.g., in Coligan, et al. (eds.) (1995 and periodic supplements) Current Protocols in Protein Science John Wiley & Sons, New York, N.Y.; and Ausubel, et al (eds.) (1987 and periodic supplements) Current Protocols in Molecular Biology, Greene/Wiley, New York, N.Y. Naturally folded or denatured material can be used, as appropriate, for producing antibodies. Either monoclonal or polyclonal antibodies may be generated, e.g., for subsequent use in immunoassays to measure the protein, or for immunopurification methods.

Methods of producing polyclonal antibodies are well known to those of skill in the art. Typically, an immunogen, preferably a purified protein, is mixed with an adjuvant and animals are immunized with the mixture. The animal's immune response to the immunogen preparation is monitored by taking test bleeds and determining the titer of reactivity to the protein or peptide of interest. For example, when appropriately high titers of antibody to the immunogen are obtained, usually after repeated immunizations, blood is collected from the animal and antisera are prepared. Further fractionation of the antisera to enrich for antibodies reactive to the protein can be performed, if desired. See, e.g., Harlow and Lane Antibodies, A Laboratory Manual; or Coligan (ed.) Current Protocols in Immunology. Immunization can also be performed through other methods, e.g., DNA vector immunization. See, e.g., Wang, et al. (1997) Virology 228:278-284.

Monoclonal antibodies may be obtained by various techniques familiar to those skilled in the art. Typically, spleen cells from an animal immunized with a desired antigen are immortalized, commonly by fusion with a myeloma cell. See, Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) Cell and Tissue Culture: Laboratory Procedures, John Wiley and Sons, New York, N.Y.. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies or binding compositions, including binding fragments and single chain versions, against predetermined fragments of CCR6 proteins can be raised by immunization of animals with conjugates of the fragments with carrier proteins as described above. Monoclonal antibodies are prepared from cells secreting the desired antibody. These antibodies can be screened for binding to normal or defective CCR6 protein. These monoclonal antibodies will usually bind with at least a K_{D} of about 1 mM, more usually at least about 300 μM, typically at least about 10 μM, more typically at least about 30 μM, preferably at least about 10 μM, and more preferably at least about 3 μM or better.

In some instances, it is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) Basic and Clinical Immunology (4th ed.) Lange Medical Publications, Los Altos, Calif., and references cited therein; Harlow and Lane (1988) Antibodies: A Laboratory Manual CSH Press; Goding (1986) Monoclonal Antibodies: Principles and Practice (2d ed.) Academic Press, New York, N.Y.; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve selection of libraries of antibodies in phage or similar vectors. See, e.g., Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, 10 a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemillumninescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see, Cabilly, U.S. Pat. No. 4,816,567; and Queen, et al. (1989) Proc. Nat'l Acad. Sci. USA 86:10029-10033; or made in transgenic mice, see Mendez, et al. (1997) Nature Genetics 15:146-156.

Antibody binding compounds, including binding fragments, of this invention can have significant diagnostic or therapeutic value. They can be useful as non-neutralizing binding compounds and can be coupled to toxins or radionuclides so that when the binding compound binds to the antigen, a cell expressing it, e.g., on its surface, is killed. Further, these binding compounds can be conjugated to drugs or other therapeutic agents, either directly or indirectly by means of a linker, and may effect drug targeting.

B. Other Molecules

Antibodies are merely one form of specific binding compositions. Other binding compositions, which will often have similar uses, include molecules that bind with specificity to a CCR6 receptor or its ligand, e.g., in a binding partner-binding partner fashion, an antibody-antigen interaction, or in a natural physiologically relevant protein-protein interaction, either covalent or non-covalent, e.g., proteins which specifically associate with a CCR6 protein. The molecule may be a polymer, or chemical reagent. A functional analog may be a protein with structural modifications, or may be a structurally unrelated molecule, e.g., which has a molecular shape which interacts with the appropriate binding determinants.

Drug screening using antibodies or CCR6 or fragments thereof can be performed to identify compounds having binding affinity to CCR6, or can block the natural interaction with ligand. Subsequent biological assays can then be utilized to determine if the compound has intrinsic blocking activity and is therefore an antagonist. Likewise, a compound having intrinsic stimulating activity can signal to the cells via the CCR6 and is thus an agonist in that it simulates the activity of a ligand.

As indicated above, the only known natural ligand for the CCR6 chemokine receptor is MIP-3α [Rossi et al., 1997, J. Immunology 158:1033-1036]. Mutein antagonists may be developed which maintain receptor binding but lack signaling. Structural studies of the ligand will also lead to design of new variants, particularly analogs exhibiting agonist or antagonist properties on the receptor. This can be combined with previously described screening methods to isolate muteins exhibiting desired spectra of activities.

As receptor specific binding molecules are provided, also included are small molecules identified by screening procedures. In particular, it is well known in the art how to screen for small molecules which interfere, e.g., with ligand binding to the receptor, often by specific binding to the receptor and blocking of binding by natural ligand. See, e.g., Meetings on High Throughput Screening, International Business Communications, Southborough, Mass. 01772-1749. Such molecules may compete with natural ligands, and selectively bind to the CCR6. Such specific binding compounds may be labeled or conjugated to toxic reagents.

VIII. Uses

Mammalian CCR6 and MIP-3α reagents will have a variety of therapeutic uses for, e.g., the treatment of conditions or diseases in which dysregulation of lymphocyte homeostasis has been implicated. These would include, e.g., mucosal inflammation of the gut or lung, including conditions such as allergy and asthma, inflammatory bowel disease, and ciliac disease.

Preferably, an administration regimen maximizes the amount of agonist or antagonist delivered to the patient consistent with an acceptable level of side effects. Accordingly, the amount of agonist or antagonist delivered depends in part on the particular agonist or antagonist and the severity of the condition being treated. Guidance in selecting appropriate doses is found in the literature on therapeutic uses of antibodies, e.g. Bach et al., chapter 22, in Ferrone et al., (eds.) (1985), Handbook of Monoclonal Antibodies Noges Publications, Park Ridge, N.J.; and Russell, pgs. 303-357, and Smith et al., pgs. 365-389, in Haber, et al. (eds.) (1977) Antibodies in Human Diagnosis and Therapy, Raven Press, New York, N.Y.

Determination of the appropriate dose is made by the clinician, e.g., using parameters or factors known in the art to affect treatment or predicted to affect treatment. Generally, the dose begins with an amount somewhat less than the optimum dose and it is increased by small increments thereafter until the desired or optimum effect is achieved relative to any negative side effects. Preferably, the CCR6 antibody or binding composition thereof that will be used is derived from the same species as the animal targeted for treatment, thereby minimizing a humoral response to the reagent.

The total weekly dose ranges for antibodies or fragments thereof, which specifically bind to CCR6 or MIP-3α, range generally from about 1 ng, more generally from about 10 ng, typically from about 100 ng; more typically from about 1 μg, more typically from about 10 μg, preferably from about 100 μg, and more preferably from about 1 mg per kilogram body weight. Although higher amounts may be more efficacious, the lower doses typically will have fewer adverse effects. Generally the range will be less than 100 mg, preferably less than about 50 mg, and more preferably less than about 25 mg per kilogram body weight.

The weekly dose ranges for antagonists, e.g., antibody, binding fragments, range from about 10 μg, preferably at least about 50 μg, and more preferably at least about 100 μg per kilogram of body weight. Generally, the range will be less than about 1000 μg, preferably less than about 500 μg, and more preferably less than about 100 μg per kilogram of body weight.

Dosages are on a schedule which effects the desired treatment and can be periodic over shorter or longer term. In general, ranges will be from at least about 10 μg to about 50 mg, preferably about 100 μg to about 10 mg per kilogram body weight.

Other antagonists of the ligands, e.g., muteins, are also contemplated. Hourly dose ranges for muteins range from at least about 10 μg, generally at least about 50 μg, typically at least about 100 mg, and preferably at least 500 mg per hour. Generally the dosage will be less than about 100 mg, typically less than about 30 mg, preferably less than about 10 mg, and more preferably less than about 6 mg per hour. General ranges will be from at least about 1 μg to about 1000 μg, preferably about 10 μg to about 500 μg per hour.

The phrase “effective amount” means an amount sufficient to modulate or ameliorate a symptom, or time of onset of symptom, typically by at least about 10%; usually by at least about 20%, preferably at least about 30%, or more preferably at least about 50%. Typical mammalian hosts will include mice, rats, cats, dogs, and primates, including humans. An effective amount for a particular patient may vary depending on factors such as the condition being treated, the overall health of the patient, the method, route, and dose of administration and the severity of side affects. When in combination, an effective amount is in ratio to a combination of components and the effect is not limited to individual components alone.

The present invention provides reagents which will find use in additional diagnostic and therapeutic applications as described elsewhere herein, e.g., in the general description for physiological or developmental abnormalities, or below in the description of kits for diagnosis. See, e.g., Berkow (ed.) The Merck Manual of Diagnosis and Therapy, Merck & Co., Rahway, N.J.; Thorn, et al. Harrison's Principles of Internal Medicine McGraw-Hill, NY; Gilman, et al. (eds. 1990) Goodman and Gilman's: The Pharmacological Bases of Therapeutics 8th Ed., Pergamon Press; (1990) Remington's Pharmaceutical Sciences (18th ed.) Mack Publishing Co., Easton, Pa.; Langer (1990) Science 249:1527-1533; Merck Index, Merck & Co., Rahway, N.J.; Avis, et al. (eds. 1993) Pharmaceutical Dosage Forms: Parenteral Medications 2d ed., Dekker, NY; Lieberman, et al. (eds. 1990) Pharmaceutical Dosage Forms: Tablets 2d ed., Dekker, NY; Lieberman, et al. (eds. 1990) Pharmaceutical Dosage Forms: Disperse Systems Dekker, NY; Fodor, et al. (1991) Science 251:767-773, Coligan Current Protocols in Immunology; Hood, et al. Immunology Benjamin/Cummings; Paul (ed.) Fundamental Immunology; Methods in Enzymology Academic Press; Parce, et al. (1989) Science 246:243-247; Owicki, et al. (1990) Proc. Nat'l Acad. Sci. USA 87:4007-4011; and Blundell and Johnson (1976) Protein Crystallography, Academic Press, New York; each of which is incorporated herein by reference. Additional uses will be apparent to a person having ordinary skill in the art in light of the teachings provided herein.

IX. Kits

This invention also contemplates use of CCR6 and MIP-3α proteins, fragments thereof, peptides, and their fusion products and related reagents in a variety of diagnostic kits and methods for detecting the presence of a binding composition as described in, e.g., Harlow and Lane (1988) Antibodies: A Laboratory Manual CSH; U.S. Pat. No. 3,645,090; U.S. Pat. No. 3,940,475; Rattle, et al. (1984) Clin. Chem. 30:1457-1461; U.S. Pat. No. 4,659,678; and Viallet, et al. (1989) Progress in Growth Factor Res. 1:89-97; each of which is incorporated herein by reference.

The broad scope of this invention is best understood with reference to the following examples, which are not intended to limit the invention to specific embodiments.

### EXAMPLE I

### General Methods

Some of the standard methods are described or referenced, e.g., in Maniatis, et al. (1982) Molecular Cloning: A Laboratory Manual Cold Spring Harbor Laboratory, Cold Spring Harbor Press; Sambrook, et al. (1989) Molecular Cloning: A Laboratory Manual (2d ed.) vols. 1-3, CSH Press, NY; Ausubel, et al. (1987 and Supplements) Current Protocols in Molecular Biology, Greene/Wiley, New York; or Innis, et al. (eds. 1990) PCR Protocols: A Guide to Methods and Applications Academic Press, N.Y. Methods for protein purification include such methods as ammonium sulfate precipitation, column chromatography, electrophoresis, centrifugation, crystallization, and others. See, e.g., Ausubel, et al. (1987 and periodic supplements); Coligan, et al. (eds. 1995 and periodic supplements) Current Protocols in Protein Science Wiley & Sons; Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) QIAexpress: The High Level Expression & Protein Purification System QUIAGEN, Inc., Chatsworth, Calif.

FACS analyses are described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

### EXAMPLE II

### Isolation of a DNA Clone Encoding CCR6 Protein

A portion of the murine cDNA was cloned from mouse spleen cDNA (Clontech) using the following degenerate primers:

5-tayathgcnathgtnca-3 (SEQ ID NO:1) and

5-ggrttnaarcarcartg-3 (SEQ ID NO:2).

The PCR product was subcloned into the cloning vector pTA (Clontech), and sequenced. Based on this sequence, primers

DP82 (5-GTTGACCGCAGTCACGAGGAGGA-3) (SEQ ID NO:3) and

DP83 (5-CAGGATCGTGATGTCTGTGAGCCA-3) (SEQ ID NO:4)

were designed and used in a PCR reaction with Clontech RACE-ready cDNA from murine spleen. These PCR products were cloned into the pTA vector and the DNAs were sequenced using primers internal to the original CCR6 clone, and with primers corresponding to the vector. The sequence of these DNAs were compiled to generate the full-length murine CCR6 cDNA.

Standard PCR techniques can be used to amplify a CCR6 gene sequence from genomic DNA or a CCR6 or fragment from cDNA derived from mRNA. Appropriate primers are selected from the sequences described, and a full length clone is isolated. Various combinations of primers, of various lengths and possibly with differences in sequence, may be prepared. The full length clone can be used as a hybridization probe to screen for other homologous genes using stringent or less stringent hybridization conditions.

In another method, oligonucleotides can be used to screen a library. In combination with polymerase chain reaction (PCR) techniques, synthetic oligonucleotides in appropriate orientations are used as primers to select correct clones from a library.

### EXAMPLE III

### Preparation of Antibodies Specific for CCR6

Inbred Balb/c mice are immunized, e.g., with 1 ml of purified CCR6 emulsified in Freund's complete adjuvant on day 0, and in Freund's incomplete adjuvant on days 15 and 22. The mice are boosted with 0.5 ml of purified CCR6 administered intravenously.

Hybridomas are created, e.g., using the non-secreting myeloma cells line SP2/0-Ag8 and polyethylene glycol 1000 (Sigma, St. Louis, Mo.) as the fusing agent. Hybridoma cells are placed in a 96-well Falcon tissue culture plate (Becton Dickinson, NJ) and fed with DMEM F12 (Gibco, Gaithersburg, Md.) supplemented with 80 μg/ml gentamycin, 2 mM glutamine, 10% horse serum (Gibco, Gaithersburg, Md.), 1% ADCM (CRTS, Lyon, France) 10⁻⁵ M azaserine (Sigma, St. Louis, Mo.) and 5×10⁻⁵ M hypoxanthine. Hybridoma supernatants are screened for antibody production against CCR6, e.g., by immunocytochemistry (ICC) using acetone fixed CCR6 transfected COS-7 cells and/or by ELISA using CCR6 purified from COS-7 supernatants as a coating antigen. Aliquots of positive cell clones are expanded for 6 days and cryopreserved as well as propagated in ascites from pristane (2,6,10,14-tetramethylpentadecane, Sigma, St. Louis, Mo.) treated Balb/c mice who had received on intraperitoneal injection of pristane 15 days before. About 10⁵ hybridoma cells in 1 ml of PBS are given intraperitoneally, and 10 days later, ascites are collected from each mouse.

After centrifugation of the ascites, the antibody fraction may be isolated by ammonium sulfate precipitation and anion-exchange chromatography on a Zephyr-D silicium column (IBF Sepracor) equilibrated with 20 mM Tris pH 8.0. Proteins are eluted with a NaCl gradient (ranging from 0 to 1 M NaCl). 2 ml fractions may be collected and tested by ELISA for the presence of anti-CCR6 antibody. The fractions containing specific anti-CCR6 activity are pooled, dialyzed, and frozen.

### EXAMPLE IV

### Preparation of Antibodies Specific for MIP-3α

Inbred Balb/c mice are immunized, e.g., with 1 ml of purified MIP-3α emulsified in Freund's complete adjuvant on day 0, and in Freund's incomplete adjuvant on days 15 and 22. The mice are boosted with 0.5 ml of purified MIP-3α administered intravenously.

Hybridomas are created, e.g., using the non-secreting myeloma cells line SP2/0-Ag8 and polyethylene glycol 1000 (Sigma, St. Louis, Mo.) as the fusing agent. Hybridoma cells are placed in a 96-well Falcon tissue culture plate (Becton Dickinson, NJ) and fed with DMEM F12 (Gibco, Gaithersburg, Md.) supplemented with 80 μg/ml gentamycin, 2 mM glutamine, 10% horse serum (Gibco, Gaithersburg, Md.), 1% ADCM (CRTS, Lyon, France) 10⁻⁵ M azaserine (Sigma, St. Louis, Mo.) and 5×10⁻⁵ M hypoxanthine. Hybridoma supernatants are screened for antibody production against MIP-3α, e.g., by immunocytochemistry (ICC) using acetone fixed MIP-3α transfected COS-7 cells and/or by ELISA using MIP-3α purified from COS-7 supernatants as a coating antigen. Aliquots of positive cell clones are expanded for 6 days and cryopreserved as well as propagated in ascites from pristane (2,6,10,14-tetramethylpentadecane, Sigma, St. Louis, Mo.) treated Balb/c mice who had received on intraperitoneal injection of pristane 15 days before. About 10⁵ hybridoma cells in 1 ml of PBS are given intraperitoneally, and 10 days later, ascites are collected from each mouse.

After centrifugation of the ascites, the antibody fraction may be isolated by ammonium sulfate precipitation and anion-exchange chromatography on a Zephyr-D column (IBF Sepracor) equilibrated with 20 mM Tris pH 8.0. Proteins are eluted with a NaCl gradient (ranging from 0 to 1 M NaCl). 2 ml fractions may be collected and tested by ELISA for the presence of anti- MIP-3α antibody. The fractions containing specific anti-MIP-3α activity are pooled, dialyzed, and frozen.

### EXAMPLE V

### Preparation of a CCR6 Deletion Mouse

To make CCR6 knockout (KO) mice, 1-C bacterial artificial chromosome (BAC) library of 129 mouse genomic DNA was screened using the DNA fragment generated by PCR using the primers DP 82 (SEQ ID NO: 3) and DP 83 (SEQ ID NO: 4). An 8 kb Hind III fragment containing the CCR6 gene was subcloned from this library. A 1.1 kb fragment of CCR6 5′ flanking DNA and 5 kb fragment of 3′ flanking DNA were placed adjacent to a neomycin resistance gene in a cassette containing the herpes simplex virus (HSV) thymidine kinase (TK) gene (S. L. Mansour, K. R. Thomas, M. R. Capecchi, Nature (London) 336, 348 (1988). DNAs from colonies resistant to both G418 (neomycin) and ganciclovir were screened in a PCR assay using primers corresponding to the neomycin resistance gene and a region just upstream of the 5 region of homology. Targeted ES cell clones were confirmed by Southern blot analysis using a probes corresponding to the 5 and 3 regions of the CCR6 gene, and injected into blastocysts from C57BL/6 mice as described in (Manipulating the Mouse Embryo: A Laboratory Manual, (1st and 2nd editions, Cold Spring Harbor Laboratory Press, Cold Spring Harbor, N.Y., 1986, 1994).

### EXAMPLE VI

### Initial Observations on CCR6 −/− (Knockout) Mice

Analysis of CCR6 −/− vs wild type mice revealed no microscopic abnormalities. Analysis of the lymphoid content of the small intestine revealed an increase in the number of T lymphocytes, and a decrease in the number of B lymphocytes. Oral immunization of the CCR6 −/− mice with either rotavirus or KLH revealed that they had a decrease in the number of responding B cells and in the level of antigen-specific IgA immunoglobulin. This data suggests that disruption of CCR6 expression in Peyer's patches results in decreased humoral response in the gut, and an increase in resident intestinal T cells.

Aerosol challenge of the CCR6 −/− mice with ovalbumin in an allergic model of pulmonary inflammation revealed that they had a decreased infiltration of eosinophils, but had a normal IgE response. This suggests that CCR6 is an important regulator of allergen-mediated eosinophil infiltration in the lung. Other, system immune responses, such as the response to subcutaneously administered KLH were normal, suggesting that CCR6 is specifically involved in mucosal immunity.

All references cited herein are incorporated herein by reference to the same extent as if each individual publication or patent application was specifically and individually indicated to be incorporated herein by reference.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method of modulating the trafficking or activation of a lymphocyte in an animal, said method comprising contacting leukocytes in said animal with a therapeutic amount of:
a) an agonist of a mammalian CCR6 receptor;
b) an antagonist of a mammalian CCR6 receptor.
c) an agonist of a mammalian MIP-3α protein; or
d) an antagonist of a mammalian MIP-3α protein.

2. The method of claim 1, wherein the mammalian CCR6 or MIP-3α is a primate protein.

3. The method of claim 1, wherein the antagonist is an antibody which binds to said mammalian CCR6 or MIP-3α.

4. The method of claim 1, wherein the antagonist is a small molecule inhibitor.

5. The method of claim 1, wherein said animal exhibits signs or symptoms of an inflammatory or leukoproliferative condition.

6. The method of claim 5, wherein said sign or symptom is in mucosal tissue.

7. The method of claim 1, wherein said modulating is inhibiting function of the CCR6-MIP-3α interaction.

8. The method of claim 7, wherein said animal is experiencing signs or symptoms of an inflammatory condition or autoimmunity; asthma; tissue specific autoimmunity; degenerative autoimmunity; rheumatoid arthritis; atherosclerosis; multiple sclerosis; delayed hypersensitivities; skin grafting; psoriasis; a transplant; spinal injury; stroke; neurodegeneration; or ischemia.

9. The method of claim 7, wherein said administering is in combination with:
a) an anti-inflammatory cytokine agonist or antagonist;
b) an analgesic;
c) an anti-inflammatory agent;
d) an anti-diarrheal agent; or
e) asteroid.

10. The method of claim 1, wherein said modulating is enhancing function of the CCR6-MIP-3α interaction.

11. The method of claim 10, wherein said administering is an agonist of CCR6 or MIP-3α.

12. The method of claim 11, wherein said animal experiences signs or symptoms of ciliac disease.

13. A genetically engineered non-human animal whose genome lacks a functional CCR6 gene.

14. The genetically engineered animal according to claim 13 wherein the animal is a rodent.

15. The genetically engineered animal according to claim 14 wherein the rodent is a mouse.

16. A genetically engineered non-human animal embryo whose somatic and germ cells lack a functional CCR6 gene.


SEQUENCE LISTING (4 sequences)

SEQ ID NO: 1 - DNA, 17 bp, Artificial Sequence
  Description of Artificial Sequence PCR primer
tayathgcnathgtnca

SEQ ID NO: 2 - DNA, 17 bp, Artificial Sequence
  Description of Artificial Sequence PCR primer
ggrttnaarcarcartg

SEQ ID NO: 3 - DNA, 23 bp, Artificial Sequence
  Description of Artificial Sequence PCR primer
gttgaccgcagtcacgaggagga

SEQ ID NO: 4 - DNA, 24 bp, Artificial Sequence
  Description of Artificial Sequence PCR primer
caggatcgtgatgtctgtgagcca